(19) United States
(12) Patent Application Publication
(10) Pub. No.: US 2026/0077149 A1
(43) Pub. Date: Mar. 19, 2026

(54) ONE-TOUCH INTEGRATED-COUPLING-TYPE MEDICAL HUMIDIFIER

(71) Applicant: SEINMEDI INC., Chilgok-gun Gyeongsangbuk-do (KR)

(72) Inventors: Ji Won SEOK, Chilgok-gun Gyeongsangbuk-do (KR); In Ho JEONG, Chilgok-gun Gyeongsangbuk-do (KR); Soung Woog SHIN, Cheonan-si Chungcheongnam-do (KR); Soon Gyun MOON, Changwon-si Gyeongsangnam-do (KR)

(21) Appl. No.: 19/401,716

(22) Filed: Nov. 26, 2025

Related U.S. Application Data
(63) Continuation of application No. PCT/KR2024/004896, filed on Apr. 12, 2024.

(30) Foreign Application Priority Data
Jun. 15, 2023 (KR) 10-2023-0077055

(51) Int. Cl. A61M 16/16

(52) U.S. Cl. CPC A61M 16/16

(57) ABSTRACT

A one-touch integrated-coupling-type medical humidifier may include a humidification container accommodating a fluid and having an open upper portion, a cap coupled to the open upper portion of the humidification container, and a gas pipe coupled to a lower portion of the cap and inserted into the humidification container, in which the humidification container includes a first coupling part protruding upward and having a closed curve-shaped cross-section, and in which the cap includes a second coupling part protruding downward and fitted with and coupled to the first coupling part.

### TECHNICAL FIELD

The present disclosure relates to a one-touch integrated-coupling-type medical humidifier, and more particularly, to a one-touch integrated-coupling-type medical humidifier in which coupling time is shortened through one-touch fastening of a connector, a cap, and a humidification container such that convenience of use is increased, internal contamination, which may occur during a coupling process, is minimized through structural integration such that safety of use is improved, disposable use is induced through structural coupling that induces breakage during separation after fastening, and thus convenience of use and safety of use may be simultaneously improved.

### BACKGROUND ART

In general, an oxygen flowmeter-coupled humidifier or a medical humidifier, which is used for oxygen inhalation treatment for patients in hospital emergency rooms, intensive care units, wards, ambulances, and the like, is supplied with gaseous dry oxygen from a terminal end of a medical gas piping facility or a portable oxygen cylinder (so-called bomb) and supplies humidified oxygen with a constant humidity to the patient through a flow meter including a flow meter part and a humidifier part.

For example, FIG. 1 illustrates an oxygen flowmeter-coupled humidifier in the related art. The oxygen flowmeter-coupled humidifier may include a flow meter part a including a flow rate gauge 31, a gauge cover 32, a flow rate adjustment mechanism 33, an adapter 34, and a body 35, and a humidifier part b including a connector 36, a humidification container 37, a duct 38, a diffuser 39, and a discharge port 40.

Gaseous oxygen, which is introduced from the outside through the adapter 34, may be controlled by the flow rate adjustment mechanism 33 and checked by the flow rate gauge 31. The introduced dry oxygen passes through the body 35 and the connector 36, passes through the cylindrical duct 38 suspended within the humidification container 37, and then generates bubbles by means of the diffuser 39 immersed in purified water 41, such that humidified oxygen is produced above a surface of the purified water 41 and supplied to the patient through the discharge port 40.

However, because the above-mentioned oxygen flowmeter-coupled humidifier in the related art is reused by replenishing purified water, there is a high risk of endotoxin and bacterial proliferation in the humidification container, which frequently results in secondary infection problems.

In addition, due to the necessity of periodic sterilization for reuse, convenience of use for medical staff and patients is significantly reduced.

Therefore, there is a need for development of a medical humidifier capable of simultaneously improving user safety and convenience of use.

### SUMMARY

### Technical Problem

The present disclosure has been made in an effort to solve the above-mentioned problem, and an object of the present disclosure is to provide a one-touch integrated-coupling-type medical humidifier in which coupling time is shortened through one-touch fastening of a connector, a cap, and a humidification container such that convenience of use is increased, internal contamination, which may occur during a coupling process, is minimized through structural integration such that safety of use is improved, disposable use is induced through structural coupling that induces breakage during separation after fastening, and thus convenience of use and safety of use may be simultaneously improved.

### Technical Solution

A one-touch integrated-coupling-type medical humidifier according to one embodiment of the present disclosure includes: a humidification container configured to accommodate therein a fluid and having an open upper portion; a cap coupled to the open upper portion of the humidification container and configured to seal the humidification container; and a gas pipe coupled to a lower portion of the cap and inserted into the humidification container, in which the humidification container includes a first coupling part protruding upward and having a closed curve-shaped cross-section, and in which the cap includes a second coupling part protruding downward and fitted with and coupled to the first coupling part.

In addition, the second coupling part may be fitted with and coupled to the first coupling part in a state in which an outer side of the second coupling part is in contact with an inner side of the first coupling part.

In addition, the first coupling part may include a protruding portion protruding along an outer peripheral surface thereof, and the protruding portion may include: a first protruding surface configured to come into contact with the lower portion of the cap; a second protruding surface extending downward from the first protruding surface and formed to be inclined; a third protruding surface extending downward from the second protruding surface and formed vertically; and a fourth protruding surface formed to be bent inward from the third protruding surface.

In addition, the second coupling part may include: an inner coupling part configured to come into contact with an inner side of the first coupling part; and an outer coupling part formed to be spaced apart from the inner coupling part and configured to come into contact with an outer side of the first coupling part.

In addition, a length of the inner coupling part in a downward direction may be longer than that of the outer coupling part.

In addition, the outer coupling part may include: an outer coupling body; a notch formed along an outer peripheral surface of the outer coupling body; and an outer catching piece formed at an end of the outer coupling body.

In addition, the outer coupling part may be provided as a plurality of outer coupling parts spaced apart from one another at predetermined intervals.

In addition, the one-touch integrated-coupling-type medical humidifier according to one embodiment of the present disclosure may further include: a connector coupled to an upper portion of the cap.

In addition, the cap may further include a connector coupling part protruding from the upper portion of the cap and inserted and coupled into the connector.

In addition, the connector coupling part may include: a coupling neck recessed along a periphery thereof; and a coupling wing part formed on an upper portion of the coupling neck and inserted into the connector.

### Advantageous Effects

According to the one-touch integrated-coupling-type medical humidifier according to the present disclosure, the coupling time may be shortened by means of one-touch fastening of the connector, the cap, and the humidification container, thereby improving the convenience of use.

In addition, internal contamination, which may occur during a coupling process, may be minimized by means of structural integration such that safety of use may be improved.

In addition, the disposable use may be induced by means of structural coupling that induces breakage when the cap and the humidification container are separated after the cap and the humidification container are fastened, thereby simultaneously improving the convenience of use and the safety of use.

### BRIEF DESCRIPTION OF DRAWINGS

FIG. 1 is a perspective view illustrating an oxygen flowmeter-coupled humidifier in the related art.

FIG. 2 is an exploded perspective view illustrating a one-touch integrated-coupling-type medical humidifier according to one embodiment of the present disclosure.

FIGS. 3A and 3B are side views illustrating a configuration in which a cap and a connector are coupled to a humidification container of the one-touch integrated-coupling-type medical humidifier according to one embodiment of the present disclosure when viewed from the lateral side, and a cross-sectional view illustrating a state in which the configuration is cut in an upward/downward direction from the lateral side.

FIGS. 4A and 4B are side views illustrating the humidification container of the one-touch integrated-coupling-type medical humidifier according to one embodiment of the present disclosure when viewed from the lateral side, and a cross-sectional view illustrating a state in which the humidification container is cut in the upward/downward direction.

FIGS. 5A and 5B are side views illustrating the cap of the one-touch integrated-coupling-type medical humidifier according to one embodiment of the present disclosure when viewed from the lateral side, and a cross-sectional view illustrating a state in which the cap is cut in the upward/downward direction.

FIGS. 6A and 6B are bottom perspective views illustrating the cap of the one-touch integrated-coupling-type medical humidifier according to one embodiment of the present disclosure when viewed from the bottom side, and a cross-sectional view illustrating a state in which the cap is cut.

FIG. 7 is a bottom plan view illustrating the cap of the one-touch integrated-coupling-type medical humidifier according to one embodiment of the present disclosure when viewed from the bottom side.

FIG. 8 is a cross-sectional view illustrating a state in which a connector of the one-touch integrated-coupling-type medical humidifier according to one embodiment of the present disclosure is cut in the upward/downward direction from the lateral side.

FIG. 9 is a cross-sectional view illustrating a state in which a gas pipe of the one-touch integrated-coupling-type medical humidifier according to one embodiment of the present disclosure is cut in the upward/downward direction from the lateral side.

FIG. 10 is an enlarged view illustrating various embodiments of a second coupling part fitted with and coupled to a first coupling part in the one-touch integrated-coupling-type medical humidifier according to the present disclosure.

FIG. 11 is a cross-sectional view illustrating a state in which a configuration in which a coupling wing part is fitted with a connector of the one-touch integrated-coupling-type medical humidifier according to another embodiment of the present disclosure is cut in the upward/downward direction from the lateral side.

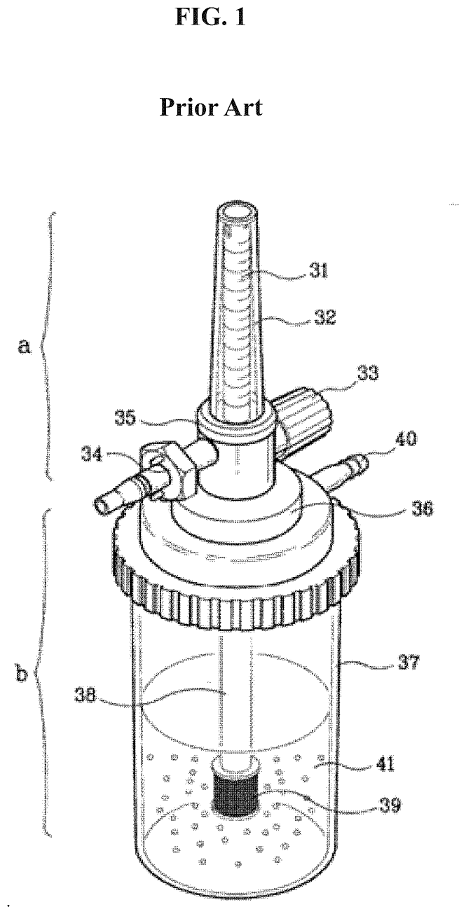

### DETAILED DESCRIPTION OF EXEMPLARY EMBODIMENTS

Hereinafter, exemplary embodiments of the present disclosure will be described in detail with reference to the accompanying drawings so that those with ordinary skill in the art to which the present disclosure pertains may easily carry out the embodiments. However, the present disclosure may be implemented in various different ways and is not limited or restricted by the embodiments described herein.

A part irrelevant to the description will be omitted to clearly describe the present disclosure. Further, the specific descriptions of publicly known related technologies will be omitted when it is determined that the specific descriptions may unnecessarily obscure the subject matter of the present disclosure. In assigning reference numerals to constituent elements of the respective drawings in the present specification, the same or similar constituent elements will be designated by the same or similar reference numerals throughout the specification.

In addition, terms or words used in the specification and the claims should not be interpreted as being limited to a general or dictionary meaning and should be interpreted as a meaning and a concept which conform to the technical spirit of the present disclosure based on a principle that an inventor can appropriately define a concept of a term in order to describe his/her own invention by the best method.

### One Embodiment

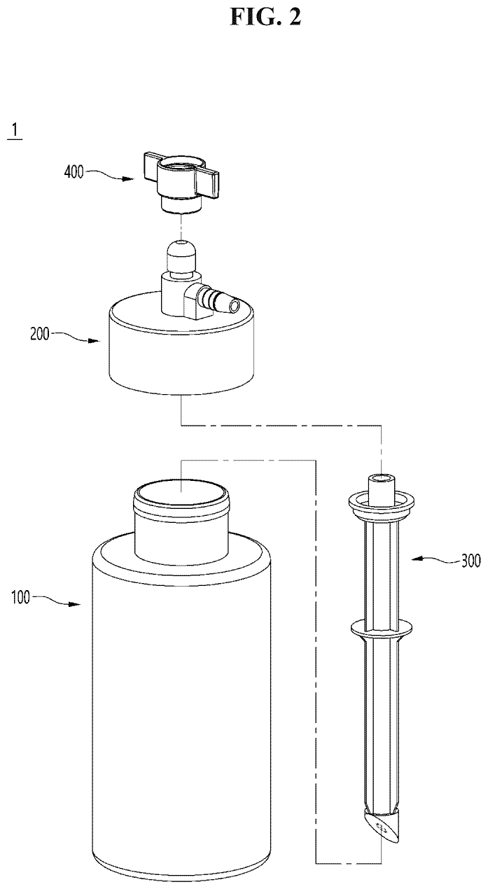

First, with reference to FIG. 2, a one-touch integrated-coupling-type medical humidifier 1 according to one embodiment of the present disclosure includes a humidification container 100, a cap 200, and a gas pipe 300.

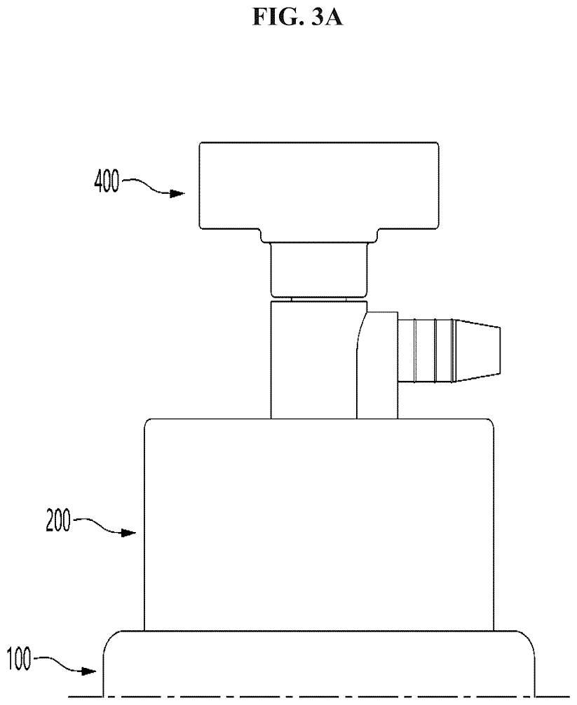

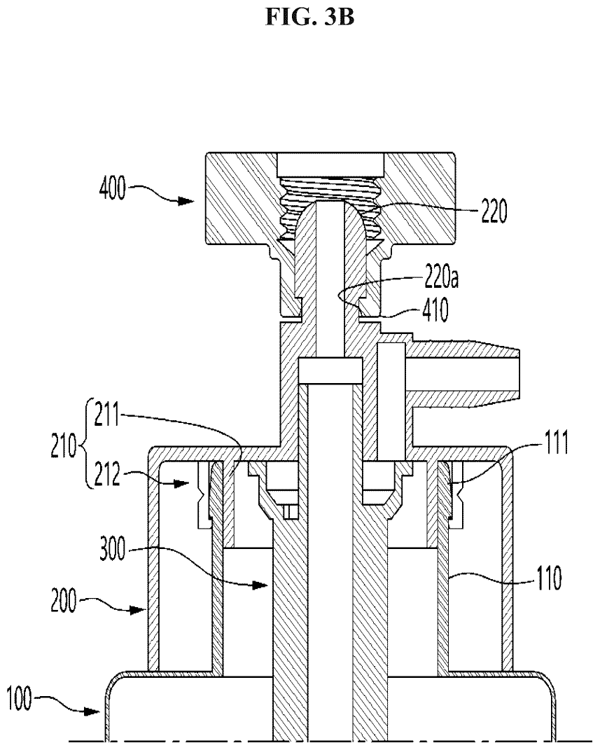

More specifically, with reference to FIGS. 2 and 3, the one-touch integrated-coupling-type medical humidifier 1 includes the humidification container 100 configured to accommodate therein a fluid and having an open upper portion, the cap 200 coupled to the open upper portion of the humidification container 100 and configured to seal the humidification container 100, and the gas pipe 300 coupled to a lower portion of the cap 200 and inserted into the humidification container 100.

In this case, the humidification container 100 includes a first coupling part 110 protruding upward and having a closed curve-shaped cross-section and includes a second coupling part 210 protruding toward the lower portion of the cap 200 and fitted with and coupled to the first coupling part 110.

Therefore, according to the present disclosure, coupling time may be shortened by means of one-touch fastening of the humidification container 100 and the cap 200 such that convenience of use may be improved, and internal contamination, which may occur during a coupling process, may be minimized by means of structural integration such that safety of use may be improved.

More specifically, a user may fit and couple the cap 200 with and to the humidification container 100 in a one-touch coupling manner instead of a method in the related art that rotates the cap 200 above the humidification container 100 and couples the cap 200, thereby innovatively shortening the coupling time, improving the convenience of use, preventing the introduction of contaminants during the process, and preventing secondary infection caused by the contaminants.

Meanwhile, as illustrated in FIG. 2, the cap 200 may include the second coupling part 210, and the second coupling part 210 may be fitted with and coupled to the first coupling part 110 in a state in which an outer side of the second coupling part 210 is in contact with an inner side of the first coupling part 110. Because the second coupling part 210 is fitted with and coupled to the first coupling part 110 in the state in which the outer side of the second coupling part 210 is in contact with the inner side of the first coupling part 110 as described above, such that a coupling force between the first coupling part 110 and the second coupling part 210 may be increased, and sealability may be improved, thereby preventing the fluid accommodated in the humidification container 100 from leaking to the outside.

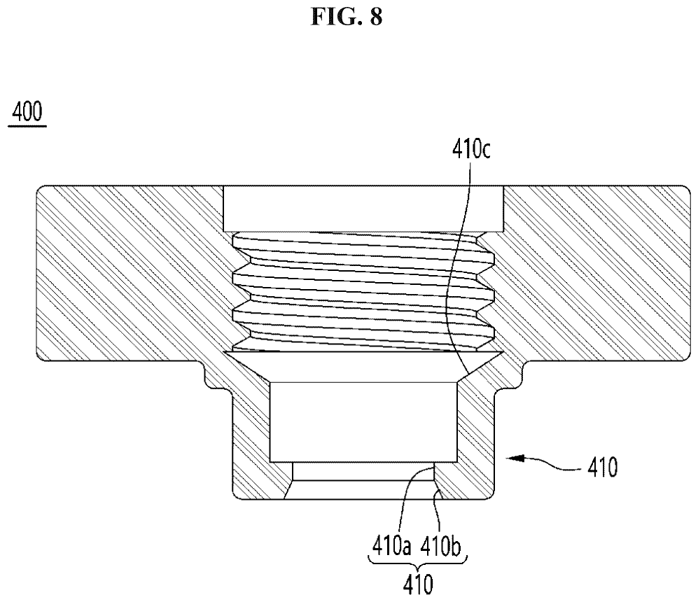

In addition, as illustrated in FIGS. 2 and 8, the one-touch integrated-coupling-type medical humidifier 1 according to one embodiment of the present disclosure may further include a connector 400 coupled to an upper portion of the cap 200. The connector 400 may serve to connect the humidification container 100 to an oxygen supplier configured to supply oxygen. Alternatively, the connector 400 may serve to connect the humidification container 100 to a flowmeter configured to identify a flow rate of oxygen, a manometer configured to visibly identify pressure in an oxygen container, and an overpressure discharge valve configured to safely discharge oxygen in case that overpressure occurs in a pressure relief valve when oxygen is supplied.

Therefore, the connector 400 may include an upper insertion groove formed in an upper portion of the connector 400, a third coupling part 410 formed on a lower portion of the connector 400, and a lower insertion groove formed in a lower surface of the third coupling part 410.

The oxygen supplier, the flowmeter, or the like may be connected to the upper insertion groove, and a connector coupling part 220 to be described below may be inserted into the lower insertion groove.

In addition, with continued reference to FIG. 8, the lower insertion groove, which is formed in the lower portion by the third coupling part 410, may be formed with a fitting guide surface 410b formed such that a diameter of the groove decreases from below to above, and a fitting close contact surface 410a formed on an upper portion of the fitting guide surface 410b and formed vertically. According to the present disclosure, the fitting guide surface 410b may be provided and serve to guide the connector coupling part 220 to be described below so that the connector coupling part 220 is easily inserted, and the fitting close contact surface 410a may be provided and serves as a catching projection after the connector coupling part 220 is tightly attached, thereby improving a coupling force between the connector 400 and the cap 200.

Meanwhile, the cap 200, the gas pipe 300, and the connector 400 may be integrally coupled and commercialized in the form of an integrated cap assembly, and the user may couple the integrated cap assembly and the humidification container 100 in a one-touch manner, thereby shortening the coupling time and minimizing internal contamination that may occur during the coupling process.

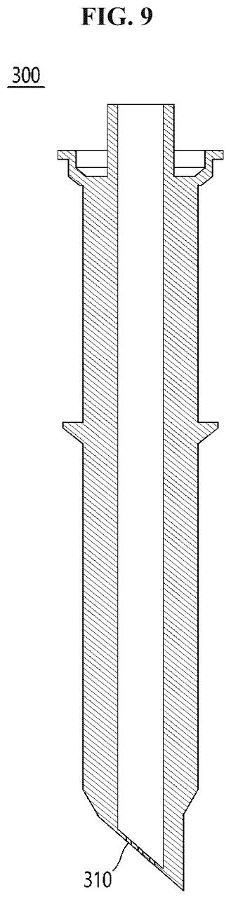

With reference to FIGS. 3 and 9, a filter 310 configured to filter out fine impurities may be provided at an end of the gas pipe 300.

Hereinafter, the components of the medical humidifier 1 according to one embodiment of the present disclosure will be described below in detail.

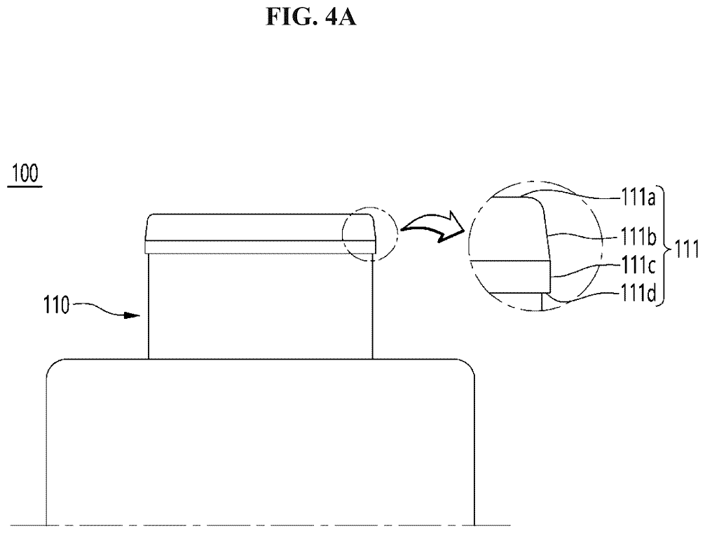

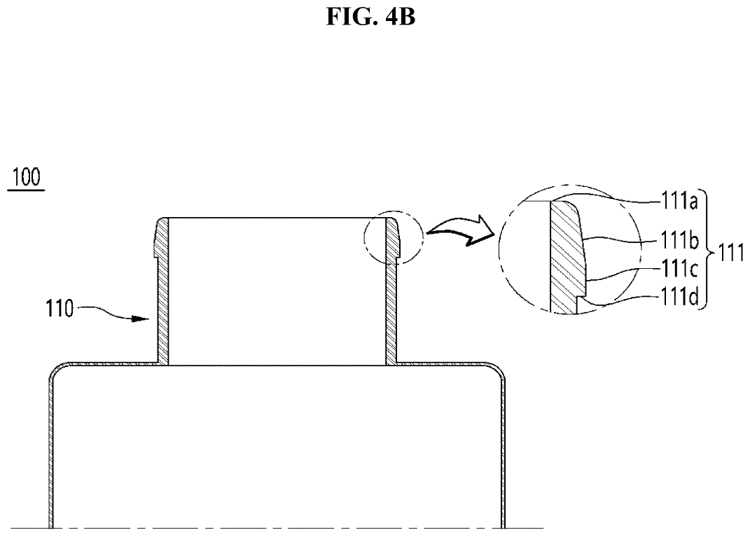

First, with reference to FIGS. 3 and 4, an accommodation space may be formed in the humidification container 100, and the fluid may be accommodated in the accommodation space. In this case, the fluid may refer to water, particularly, purified water or sterile water.

The humidification container 100 may have the open upper portion and include the first coupling part 110 protruding upward and having a closed curve-shaped cross-section as described above. In this case, the closed-curve shape may particularly be a circular shape. However, the present disclosure is not necessarily limited thereto. The first coupling part 110 may provide a coupling region in which the humidification container 100 may be coupled to the cap 200.

Continuously, the first coupling part 110 may include a protruding portion 111 protruding along an outer peripheral surface thereof. In this case, the protruding portion 111 is coupled to an outer coupling part 212 of the second coupling part 210 to be described below, such that the humidification container 100 and the cap 200 may be fitted and coupled.

More specifically, the protruding portion 111 may include a first protruding surface 111a, a second protruding surface 111b, a third protruding surface 111c, and a fourth protruding surface 111d. The first protruding surface 111a is a portion configured to come into contact with the lower portion of the cap 200. The first protruding surface 111a may be formed in the shape of a horizontal flat surface, maintain sealability between the cap 200 and the humidification container 100, and prevent the fluid in the humidification container 100 from leaking.

Next, the second protruding surface 111b may be formed to extend and be inclined downward from the first protruding surface 111a. Therefore, when the cap 200 moves downward from above, an outer catching piece 212c of the outer coupling part 212 to be described below may move downward while sliding, such that the humidification container 100 and the cap 200 may be softly fitted and coupled. In particular, a notch 212b is formed in the outer coupling part 212, such that the outer coupling part 212 is easily broken in case that an external force is applied. Because the outer catching piece 212c slides due to the second protruding surface 111b during the coupling process, it is possible to prevent the outer coupling part 212 from being inadvertently broken during the coupling process.

Next, the third protruding surface 111c may extend downward from the second protruding surface 111b and be formed vertically. The third protruding surface 111c is a portion configured to come into contact with an inner coupling part 211. The third protruding surface 111c may be formed vertically and tightly attached to the inner coupling part 211, thereby increasing the coupling force and maintaining the sealability between the cap 200 and the humidification container 100.

Lastly, the fourth protruding surface 111d may be bent inward from the third protruding surface 111c. Therefore, the outer catching piece 212c of the outer coupling part 212 and the protruding portion 111 of the first coupling part 110 may be structured to be coupled to each other and make it difficult to separate the cap 200 and the humidification container 100 after the cap 200 and the humidification container 100 are fastened. In addition, in case that the cap 200 is separated by applying an external force, which exceeds the coupling force, after the cap 200 and the humidification container 100 are coupled, the bent shape of the fourth protruding surface 111d is broken, and as a result, the cap 200 and the humidification container 100 are not coupled again, thereby preventing the reuse of the cap 200.

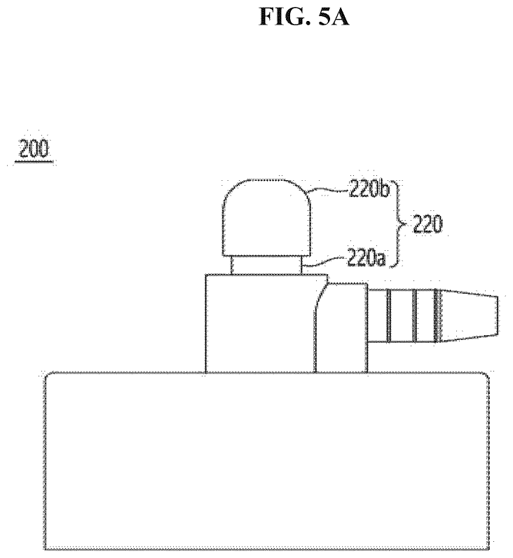

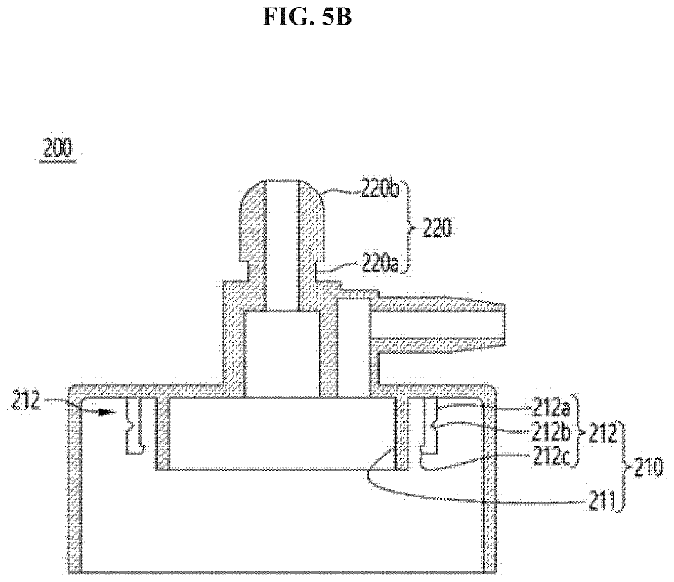

Next, the second coupling part 210 of the present disclosure will be described in detail with reference to FIGS. 3 and 5.

The second coupling part 210 may include the inner coupling part 211 configured to come into contact with the inner side of the first coupling part 110, and the outer coupling part 212 formed to be spaced apart from the inner coupling part 211 and configured to come into contact with an outer side of the first coupling part 110. In case that the outer coupling part 212 and the inner coupling part 211 are coupled to two opposite sides, i.e., the outer and inner sides of the first coupling part 110 as described above, the inner coupling part 211 may serve to support the cap 200 so that the cap 200 is not separated from the humidification container 100 even though an external force is applied in the horizontal direction after the cap 200 is fastened to the humidification container 100. Meanwhile, in this case, a distance at which the outer coupling part 212 is spaced apart from the inner coupling part 211 may correspond to a thickness of the first coupling part 110.

In addition, a length of the inner coupling part 211 in the downward direction may be longer than that of the outer coupling part 212. Therefore, a supporting force of the inner coupling part 211 may be improved, thereby further preventing the cap 200 from being separated even though a horizontal external force is applied to the cap 200 coupled to the humidification container 100.

Continuously, the outer coupling part 212 will be described in detail with reference to FIG. 5. The outer coupling part 212 may include an outer coupling body 212a, the notch 212b formed along an outer peripheral surface of the outer coupling body 212a, and the outer catching piece 212c formed at an end of the outer coupling body 212a.

The outer coupling body 212a may serve as a body that constitutes the outer coupling part 212. In addition, the notch 212b may be formed in a shape recessed along the outer peripheral surface of the outer coupling body 212a. When the humidification container 100 and the cap 200 are about to be separated by applying an external force after the humidification container 100 and the cap 200 are fitted and coupled by the first coupling part 110 and the second coupling part 210, the notch 212b may induce the outer coupling part 212 to be bent or fractured.

That is, in case that an attempt is made to separate the humidification container 100 and the cap 200 by applying an external force after the humidification container 100 and the cap 200 are coupled, the outer coupling part 212 may be easily bent or fractured by the notch 212b, and a lower region of the notch 212b may be detached, thereby preventing the first coupling part 110 and the second coupling part 210 from being coupled again. As a result, it is possible to prevent the reuse of the humidifier 1 and induce the disposable use.

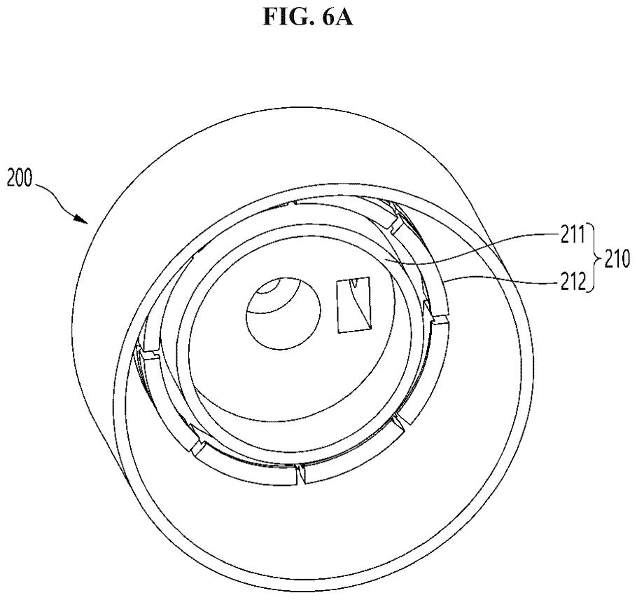

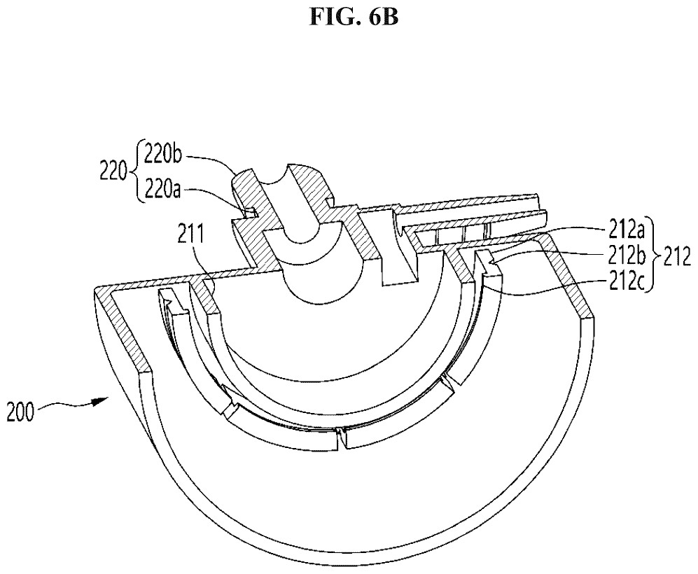

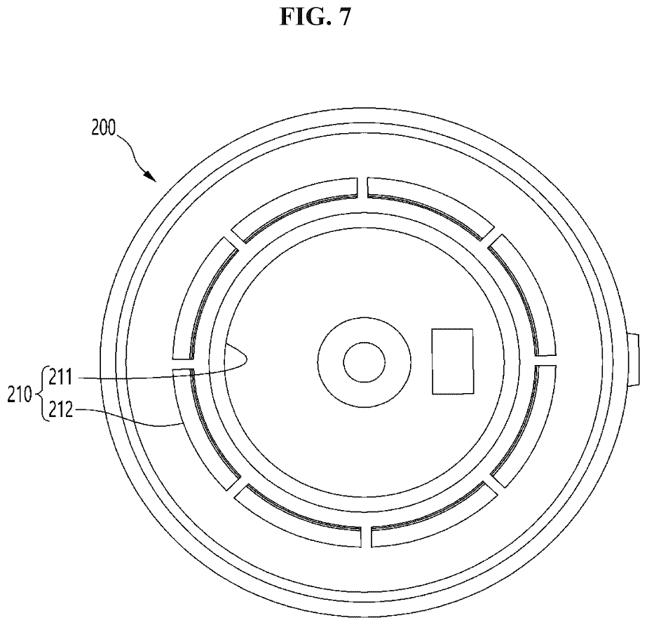

Meanwhile, with reference to FIGS. 6 and 7, in the one-touch integrated-coupling-type medical humidifier 1 according to one embodiment of the present disclosure, the outer coupling part 212 may be provided as a plurality of outer coupling parts 212 spaced apart from one another at predetermined intervals. Therefore, in case that the second coupling part 210 moves downward so as to be fitted with and coupled to the first coupling part 110, a space in which the outer coupling part 212 may be widened in an outer peripheral direction may be provided, such that the second coupling part 210 may be easily fitted with and coupled to the first coupling part 110.

However, as necessary, the outer coupling parts 212 may be integrated and connected to have a closed curve-shaped cross-section without being spaced apart from one another. In this case, it is possible to improve the effect of increasing the coupling force.

In the one-touch integrated-coupling-type medical humidifier 1 according to one embodiment of the present disclosure, the cap 200 may further include the connector coupling part 220. The connector coupling part 220 may protrude from the upper portion of the cap 200 and inserted and coupled into the connector 400. Therefore, the cap 200, the gas pipe 300, and the connector 400 may be integrally coupled and commercialized in the form of an integrated cap assembly, and the user may couple the integrated cap assembly and the humidification container 100 in a one-touch manner, thereby shortening the coupling time and minimizing internal contamination that may occur during the coupling process.

Meanwhile, the connector coupling part 220 may include a coupling neck 220a and a coupling wing part 220b.

The coupling neck 220a may be recessed along a periphery of the connector coupling part 220, and the third coupling part 410 of the connector 400 may be seated on the recessed coupling neck 220a and allow the connector 400 and the connector coupling part 220 to be coupled.

In addition, the coupling wing part 220b may be formed on an upper portion of the coupling neck 220a and inserted into the connector 400, and the coupling wing part 220b may serve to maintain sealability in the connector 400.

### Another Embodiment

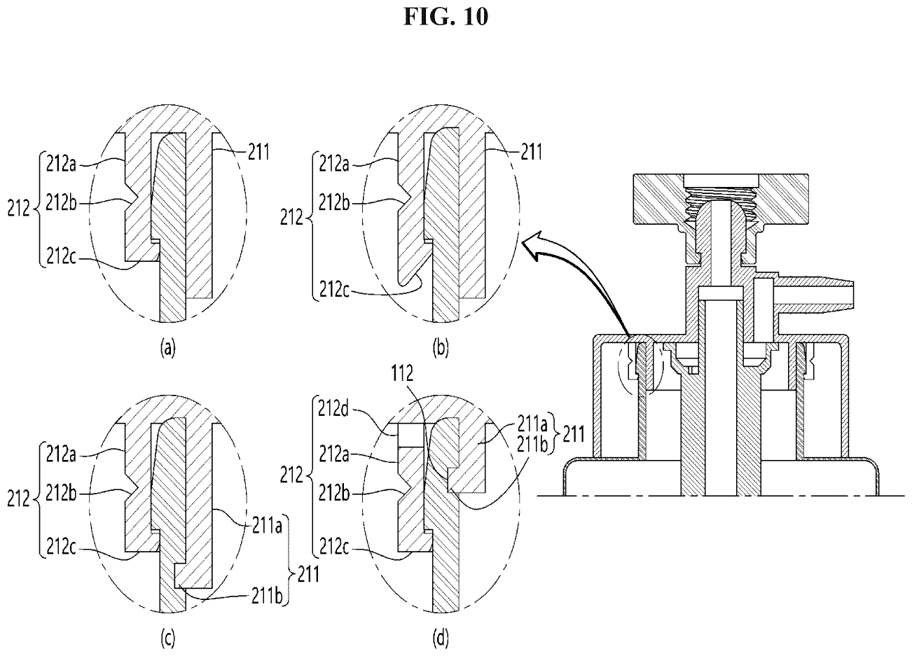

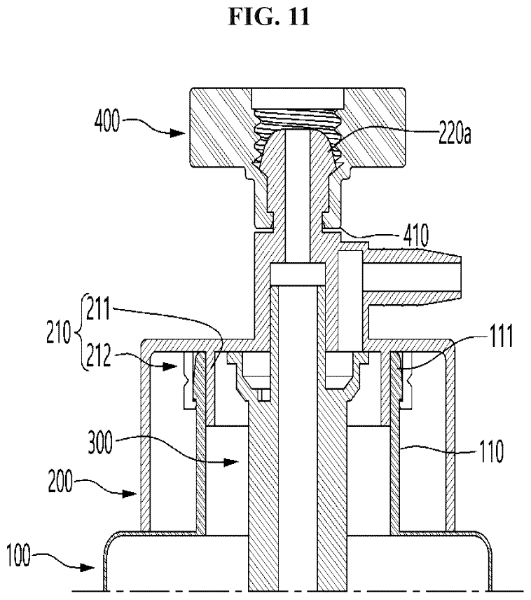

Hereinafter, another embodiment of the present disclosure will be described with reference to FIGS. 10 and 11. The components of the present disclosure, which will not be described below, may be understood as the components disclosed in one embodiment.

First, with reference to (b) of FIG. 10, the outer catching piece 212c may be formed to be inclined at a predetermined angle or formed as a curved surface having a predetermined curvature. Therefore, the first coupling part 110 and the second coupling part 210 may be easily coupled. More specifically, the first coupling part 110 and the second coupling part 210 may be coupled while sliding in a state in which the second protruding surface 111b of the first coupling part 110 and the outer catching piece 212c of the second coupling part 210 are in contact with each other.

Next, with reference to (c) of FIG. 10, the inner coupling part 211 may include an inner coupling body 211a and an inner catching piece 211b, and the inner catching piece 211b may be inserted and fitted into a coupling groove formed in the first coupling part 110, thereby increasing a coupling force.

Lastly, with reference to (d) of FIG. 10, the outer coupling part 212 may further include a tear-off piece 212d formed in a region connected directly to a lower end of the cap 200. The tear-off piece 212d may be cut off by a horizontal rotation of the cap 200. In this case, the outer coupling part 212 may be detached, the coupling force between the cap 200 and the humidification container 100 may be decreased, and the cap 200 and the humidification container 100 may be uncoupled. However, in case that the cap 200 and the humidification container 100, which have been uncoupled once, are intended to be coupled again, the cap 200 and the humidification container 100 cannot be coupled again because the outer coupling part 212 is detached, thereby preventing the reuse of the humidifier 1.

Lastly, with continued reference to (d) of FIG. 10, together with the above-mentioned configuration of the tear-off piece 212d, a length of the inner coupling part 211 in the upward/downward direction may be shorter than a length of the outer coupling part 212 in the upward/downward direction. Therefore, in case that a horizontal external force is applied after the tear-off piece 212d is detached, the cap 200 may be easily separated from the humidification container 100.

Meanwhile, the inner catching piece 211b having a bent shape may be further formed at an end of the inner coupling part 211, and the coupling groove may be formed in an inner peripheral surface of the protruding portion 111 of the first coupling part 110, such that the inner catching piece 211b may be inserted into the coupling groove. In this case, it is possible to improve the coupling force and sealability between the cap 200 and the humidification container 100. After the tear-off piece 212d is detached, the above-mentioned configuration may perform a minimum securing function that prevents the cap 200 and the humidification container 100 from being separated without any external force.

Meanwhile, with reference to FIG. 11, the coupling wing part 220b according to another embodiment of the present disclosure may be made of a material having elasticity and formed in a shape having a diameter that increases upward. In case that the coupling wing part 220b is made of an elastic material, the coupling wing part 220b may be easily inserted and coupled even though a diameter of the coupling wing part 220b is larger than a diameter of the lower insertion groove formed by the third coupling part 410. After the coupling wing part 220b is coupled, pressure may be applied in an inner peripheral direction of the connector 400, thereby improving the sealability and coupling force.

Furthermore, the connector 400 may further include a wing close contact surface 410c formed in an internal space and having a diameter that increases upward. Therefore, in case that the coupling wing part 220b made of an elastic material is spread after the connector coupling part 220 is inserted into the connector 400, the coupling wing part 220b may be tightly attached to the wing close contact surface 410c, thereby improving the sealability and coupling force.

The present disclosure has been described with reference to the limited embodiments and the drawings, but the present disclosure is not limited thereby. The present disclosure may be carried out in various forms by those skilled in the art, to which the present disclosure pertains, within the technical spirit of the present disclosure and the scope equivalent to the appended claims.

### Description of Reference Numerals

1: Medical humidifier
100: Humidification container
110: First coupling part
111: Protruding portion
111a: First protruding surface
111b: Second protruding surface
111c: Third protruding surface
111d: Fourth protruding surface
200: Cap
210: Second coupling part
211: Inner coupling part
211a: Inner coupling body
211b: Inner catching piece
212: Outer coupling part
212a: Outer coupling body
212b: Notch
212c: Outer catching piece
212d: Tear-off piece
220: Connector coupling part
220a: Coupling neck
220b: Coupling wing part
300: Gas pipe
310: Filter
400: Connector
410: Third coupling part
410a: Fitting close contact surface
410b: Fitting guide surface
410c: Wing close contact surface

1. A one-touch integrated-coupling-type medical humidifier comprising:
a humidification container configured to accommodate therein a fluid and having an open upper portion;
a cap coupled to the open upper portion of the humidification container and configured to seal the humidification container; and
a gas pipe coupled to a lower portion of the cap and inserted into the humidification container,
wherein the humidification container comprises a first coupling part protruding upward and having a closed curve-shaped cross-section, and
wherein the cap comprises a second coupling part protruding downward and fitted with and coupled to the first coupling part.

2. The one-touch integrated-coupling-type medical humidifier of claim 1, wherein the second coupling part is fitted with and coupled to the first coupling part in a state in which an outer side of the second coupling part is in contact with an inner side of the first coupling part.

3. The one-touch integrated-coupling-type medical humidifier of claim 1, wherein the first coupling part comprises a protruding portion protruding along an outer peripheral surface thereof, and
wherein the protruding portion comprises:
a first protruding surface configured to come into contact with the lower portion of the cap;
a second protruding surface extending downward from the first protruding surface and formed to be inclined;
a third protruding surface extending downward from the second protruding surface and formed vertically; and
a fourth protruding surface formed to be bent inward from the third protruding surface.

4. The one-touch integrated-coupling-type medical humidifier of claim 1, wherein the second coupling part comprises:
an inner coupling part configured to come into contact with an inner side of the first coupling part; and
an outer coupling part formed to be spaced apart from the inner coupling part and configured to come into contact with an outer side of the first coupling part.

5. The one-touch integrated-coupling-type medical humidifier of claim 4, wherein a length of the inner coupling part in a downward direction is longer than that of the outer coupling part.

6. The one-touch integrated-coupling-type medical humidifier of claim 4, wherein the outer coupling part comprises:
an outer coupling body;
a notch formed along an outer peripheral surface of the outer coupling body; and
an outer catching piece formed at an end of the outer coupling body.

7. The one-touch integrated-coupling-type medical humidifier of claim 4, wherein the outer coupling part is provided as a plurality of outer coupling parts spaced apart from one another at predetermined intervals.

8. The one-touch integrated-coupling-type medical humidifier of claim 1, further comprising:
a connector coupled to an upper portion of the cap.

9. The one-touch integrated-coupling-type medical humidifier of claim 8, wherein the cap further comprises a connector coupling part protruding from the upper portion of the cap and inserted and coupled into the connector.

10. The one-touch integrated-coupling-type medical humidifier of claim 8, wherein the connector coupling part comprises:
a coupling neck recessed along a periphery thereof; and
a coupling wing part formed on an upper portion of the coupling neck and inserted into the connector.
